(19) United States
(12) Patent Application Publication
(10) Pub. No.: US 2016/0274107 A1
(43) Pub. Date: Sep. 22, 2016

(54) METHOD OF STUDYING REACTIONS BETWEEN TWO OR MORE MOLECULES IN A BIOLOGICAL SAMPLE

(71) Applicants: Laurent COGNET, Bordeaux (FR); Brahim LOUNIS, Pessac (FR)

(72) Inventors: Laurent Cognet, Bordeaux (FR); Brahim Lounis, Pessac (FR)

(21) Appl. No.: 15/029,523

(22) PCT Filed: Oct. 14, 2014
(86) PCT No.: PCT/EP2014/071997

§ 371 Date: Apr. 14, 2016

Related U.S. Application Data
(60) Provisional application No. 61/890,371, filed on Oct. 14, 2013.

(51) Int. Cl. G01N 33/566; G02B 21/16; G01N 33/58; G01N 21/64

(52) U.S. Cl. CPC G01N 33/566; G01N 21/6458; G01N 21/6428; G02B 21/16; G01N 33/582; G01N 2021/6439; G01N 2201/12; G01N 2333/71

(57) ABSTRACT

The present invention is related to a method of studying (i) binding reactions, (ii) dissociation reactions, (iii) competitive binding reactions, or (iv) association reactions between two or more molecules in a biological sample by means of super-resolution imaging, in which method the reaction as such causes an event that forms the basis for the identification and localization-based super-resolution microscopy of the reaction (FIG. 1a).

The present invention relates to a method of studying reactions between two or more molecules in a biological sample according to the preamble of claim 1.

Molecular interactions are key to many chemical and biological processes like protein function. In many signaling processes they occur in sub-cellular areas displaying nanoscale organizations and involving molecular assemblies. The nanometric dimensions and the dynamic nature of the interactions make their investigations complex in live cells.

By providing optical images with spatial resolutions below the diffraction limit, super-resolution fluorescence microscopies opened the possibility to study biological structures with finer details compared to conventional light microscopies.

Most existing methods rely on the optical control of the nano-emitters fluorescent state population. For instance, localization-based methods consist in recovering the positions of numerous single molecules with high accuracy from image sequences where each frame contains only a few stochastically photoactivated emitters.

Several of these methods are suitable for imaging live bio-samples, giving unique details about spatio-temporal molecular organizations at the scale of a few tens of nanometers in cells.

While super-resolution fluorescence microscopies offer live-cell molecular imaging with sub-wavelength resolutions, they lack, however, specificity for distinguishing interacting molecule populations.

Functional signaling molecules are frequently highly organized in molecular assemblies. Molecular interactions occur at the nanometer scale and are generally highly dynamic. They are often triggered by an external activating signal like a specific ligand binding.

Although of prime importance to unravel several key molecular processes, the identification in super-resolved imaging of molecular assemblies like multimers, is still lacking.

### SUMMARY OF THE PRESENT INVENTION

It is one object of the present invention to provide methods which allow the identification of molecular assemblies, of their formation and/or dissociation, on a super-resolved optical magnification scale.

It is one other object of the present invention to provide methods which allow live-cell molecular imaging with sub-wavelength resolutions which are capable of distinguishing populations of interacting molecules.

It is one other object of the present invention to provide methods which allow live-cell molecular imaging with sub-wavelength resolutions of a molecular interaction/dissociation triggered by a binding reaction event.

These and other objects are solved by the subject matter of the present invention.

### EMBODIMENTS OF THE INVENTION

Before the invention is described in detail, it is to be understood that this invention is not limited to the particular component parts of the compounds described or process steps of the methods described as such compounds and methods may vary. It is also to be understood that the terminology used herein is for purposes of describing particular embodiments only, and is not intended to be limiting. It must be noted that, as used in the specification and the appended claims, the singular forms “a,” “an”, and “the” include singular and/or plural referents unless the context clearly dictates otherwise. It is moreover to be understood that, in case parameter ranges are given which are delimited by numeric values, the ranges are deemed to include these limitation values. It is moreover to be understood that, in case upper and lower limits of such parameter ranges are given, such limits may be used individually or in combination.

According to one aspect of the invention, a method of studying (i) binding reactions, (ii) dissociation reactions, (iii) competitive binding reactions, or (iv) association reactions (hereinafter called “the reaction” or “the reactions”) between two or more molecules in a biological sample is provided. The method works by means of super-resolution imaging, in which method the reaction as such causes an event that forms the basis for the identification and localization-based super-resolution microscopy of the reaction.

Preferably, in said method at least one molecule participating in the reaction is an endogenous molecule. As used herein, the term “endogenous molecule” relates to a molecule that is native to the biological sample, e.g., a cytokine, growth factor, transmitter, hormone, receptor, metabolite or the like, that is part of the biological sample's proteome. Such endogenous molecule can however be modified, e.g., it can be labeled with a suitable marker or optical probe, like a fluorophore, absorber, quencher, dye or nanoparticle, be fused to another peptide, like GFP, or the like.

Preferably, in said method the super-resolution imaging method is a stochastical super-resolution imaging method, preferably a “localization microscopy”-based method. In this approach, the positions of single probes capable of emitting, absorbing, scattering light under given conditions are recovered with high accuracy (below the diffraction limit) from image sequences, where each image sequence frame comprises only a few optically active probe which have stochastically bound or unbound to their counterpart, or were delivered or undelivered remotely, e.g. by optical trap local delivery or in microfluidic devices.

Preferably, said method provides information on the spatial localization of the reaction in the biological sample. This means that inter-molecular reactions can be localized, with high accuracy, on a sub cellular scale. A ligand-receptor reaction can thus for example be recorded, and attributed to a given sub-cellular compartment, e.g., a synapse, an endocellular membrane, or an extracellular membrane compartment.

Preferably, said method provides a spatial super-resolution of between ≦250 nm and ≧1 nm, more preferably, between ≦100 nm and ≧1 nm, more preferably, between ≦80 nm and ≧1 nm, more preferably, between ≦50 nm and ≧1 nm, more preferably, between ≦40 nm and ≧1 nm, more preferably, between ≦30 nm and ≧1 nm, more preferably, between ≦20 nm and ≧1 nm, and most preferably, between ≦10 nm and ≧1 nm. In optical microscopy the resolution d, can be stated as: d=λ/2NA, in which λ is the wavelength of the incident light and NA is the numerical aperture of the optical system. In practice the lowest value of d obtainable with conventional optical microscopy is thus about 250 nm at visible wavelength.

Preferably, in said method the super-resolution imaging method is based on point accumulation for imaging in nanoscale topography and/or universal point accumulation for imaging in nanoscale topography. This super-resolution imaging method is also known as “Paint” or “uPaint”, as described in Sharonov et al 2006 and Giannone et al, 2010.

Preferably, in said method the reaction as such causes an optical event, which event is recorded by a detector to form an image, and which event serves as basis for the identification and localization-based super-resolution microscopy of the reaction.

Preferably, in said method the reaction as such and/or the optical event, serves as the stochastic process necessary for the identification and localization-based super-resolution microscopy of the reaction.

Preferably, in said method the optical event is at least one selected from the group consisting of:
occurrence or cessation of light emission
change in wavelength of light emission
change in intensity of light emission
change of intensity of transillumination
change in light scattering
change in birefringence
change in polarization of light, and/or
phase shift of emitted or transmitted or reflected light.

Preferably, such event is caused by, e.g., ligand activation. Hence, the concept of imaging in real time agonist activation of single receptors allows the selective imaging of activated receptors with super-resolution.

This approach is for example described in Bobroff 1986, or Thompson et al 2002. In short, the optical event is caused by a molecule that has only a few nanometers in size, or even less, whereas the optical resolution of the microscope is about 200 nm. However, the optical event—e.g., the emission of fluorescent light—will (i) either create an image that is bigger than said 200 nm, or (ii) cause blooming on the detected image (e.g, due to well saturation in a CCD detector). In both situations, the event can be recorded by the detector (which can be either an array detector e.g. CCD or CMOS detectors, or 1D array or a point detector coupled to laser scanning), although the molecular source underlying the optical event is below the optical resolution limit. The event will thus create a detected image that has several pixels in size (for point detectors, pixels are not by the scanning steps), in which image the light intensity can be approximated, e.g., by a 2-dimensional Gaussian distribution. The correct position of the molecular source underlying the optical event can thus be calculated by curve fitting of said distribution, and subsequent analysis and maximum detection, thus enabling a very precise localization of the molecular source beyond the optical resolution of the microscope.

Preferably, in said method several subsequent image frames showing subsequent stages of the optical event are analyzed, in such way that
(a) the pointing accuracy of the molecular source underlying the optical event is further increased, and/or
(b) the movement dynamics of the molecular source underlying the optical event is analyzed.

Option (a) is particularly useful in case the molecular source underlying the optical event is static, and not moving. This may apply, e.g., to a ligand receptor reaction in which the receptor does not move. Option (b) can be used in case the molecular source underlying the optical event is moving.

Preferably, in said method the biological sample comprises live cells. Such embodiment encompasses, for example, live cells obtained from a cell culture, from a tissue sample, from a trypsinized tissue sample, from a cell suspension, from a blood sample or the like. However, the biological sample can also consist of a sample in which the cells are no longer intact and/or alive, as long as portions of the plasma membrane or endomembranes are present in the preparation, like for instance in tissue sections.

Preferably, in said method the two or more molecules participating in the reaction are selected from the group consisting of receptors or receptor ligands. As used herein, the term “receptor” defines a biological molecule, most often a protein bound to either a plasma membrane or an endomembrane, that is capable of evoking a physiological or biochemical function once a suitable ligand (also called agonist), has bound thereto. As used herein, the term “ligand” refers to a molecule, with the affinity to bind to a given receptor. A ligand can be either an agonist or an antagonist. As used herein, the term “agonist” shall encompass all molecules that have an affinity to a given receptor and are capable of evoking the respective physiological or biochemical function. As used herein, the term “antagonist” shall encompass all molecules that have an affinity to a given receptor, but no efficacy. This means, for example, that (i) upon binding of said antagonist to the receptor, no physiological or biochemical function is elicited, or a dampened or altered physiological or biochemical function is elicited, (ii) binding of said antagonist to the receptor inhibits the binding thereof to its suitable ligand, or (iii) binding of said antagonist to a receptor inhibits transformation metabolization thereof.

Preferably, in said method at least one molecule that participates in the reaction is labeled with an optical probe. Preferably, in said method the optical probe is a fluorophore, a quencher, an absorber, a scatterer, a phosphorescent and/or a phase-shifter.

Preferably, in said method the super-resolution imaging method comprises the application of structured illumination, e.g. oblique illumination.

Such oblique illumination of the sample can for example be achieved in a microscope by translating the excitation beam with respect to the axis of the objective. This allows to image those individual fluorophores only which enter the oblique excitation beam, e.g., because the ligand they are bound to binds to a cell surface receptor which is in the center of the oblique excitation beam. Thus, excitation & photobleaching of the fluorophores at distance from the cell surface and hence background noise are reduced.

As an alternative to oblique illumination, other types of structured illumination or localized illumination can be used, like optical lattices or light sheet illumination. The latter is also called “light sheet fluorescence microscopy” (LSFM), and is a fluorescence microscopy technique which, in contrast to Epi fluorescence microscopy, illuminates only a thin slice (usually a few hundred nanometers to a few micrometers) of the sample perpendicularly to the direction of observation. For illumination a laser lightsheet is used, i.e., a laser beam which is focused only in one direction (e.g. using a cylindrical lens). A second method uses a circular beam scanned in one direction to create the lightsheet. As only the actually observed section is illuminated, this method reduces the photodamage and stress induced on a living sample. Also the good sectioning capability reduces the background signal. Plane illumination, also called “selective plane illumination microscopy”, is a similar technique with corresponding advantages.

Preferably, in said method the reaction is a ligand-receptor reaction and/or a receptor-receptor association or dissociation. Association can, for example, consist of a dimerization of two receptor molecules induced by a stimulus. Dissociation can, for example, consist of a disassembly of a receptor dimer induced by a stimulus.

Preferably, in said method the reaction causes the spatial convergence of two or more fluorophores with suitable donor-acceptor characteristics, thus resulting in a Föorster energy transfer (FRET) reaction being the optical event, as e.g. described in Winckler et al, 2013, content of which is incorporated herein.

Preferably, in said method the reaction causes the spatial convergence of at least one fluorophore and at least one quencher with suitable fluorophore-quencher characteristics, thus resulting in a fluorescence quenching reaction being the optical event.

Preferably, in said method at least two ligands participating in the reaction are labeled with different optical probes.

This approach can for example be put into practice with two different types of EGFR ligands, part of which is labeled with a donor fluorophore and the other part of which is labeled with an acceptor fluorophore, Once both bind to different EGF-receptor monomers, the latter will form a receptor dimer, which then causes the spatial convergence of the donor and the acceptor, thus resulting in an optical event caused by FRET.

With such approach, receptor hornodimerization (like EGFR-EGFR, Her2-Her2), heterodimerization (e.g., EGFR-ErbB2, EGFR/IGF-IR, PDGFbetaR-EGFR, Her2-Her3, EGFR-Met) or receptor polymerization can be studied, as well as the respective monomerization, i.e. the disassembly of homo- or heterodimers, or -polymers.

Preferably, in said method at least one ligand and at least one receptor participating in the reaction are labeled with different optical probes.

This approach can for example be put into practice with the ligand being labeled with a donor fluorophore, and the receptor being labeled with an acceptor fluorophore, or vice versa. Once the ligand has bound to the receptor, a spatial convergence of the donor and the acceptor will occur, thus resulting in an optical event caused by FRET.

In this approach, single-molecule FRET signals originating from e.g. dimerized receptors can be continuously generated by using distinct fluorescent ligands and recording their receptor binding in real-time. This allows obtaining specific images of ligand-activated molecular dimers with super-resolution and further study their dynamic behavior in the early stage of their activation.

Alternatively, the ligand can be labeled with a fluorophore, and the receptor can be labeled with a quencher, or vice versa. The receptor can, e.g., be a recombinant fusion protein in such way that it comprises a genetically engineered peptide fluorophore or quencher, like GFP.

Preferably, in said method at least two receptors participating in the reaction are labeled with different optical probes.

This approach can for example be put into practice with two different types of EGF receptor monomers part of which is labeled with a donor fluorophore and the other part of which is labeled with an acceptor fluorophore. Once both are bound by a suitable EGFR ligand, they will form a receptor dimer, which then causes the spatial convergence of the donor and the acceptor, thus resulting in an optical event caused by FRET. With such approach, receptor homodimerization (like ESR-ESR, Her2-Her2), heterodimerization (e.g., EGFR-ErbB2, EGFR/IGF-IR, PDGFbetaR-EGFR, Her2-Her3, EGFR-Met) or receptor polymerization can be studied, as well as the respective monomerization, i.e. the disassembly of homo- or heterodimers, or -polymers.

Preferably, in said method at least one molecule participating in the reaction is added to the biological sample in real-time, e.g., during an experiment is recorded by means of the image detector.

Thus, the method extends beyond a mere static analysis of the binding behavior, and provides a dynamic insight into the binding process. If the added molecules are, e.g., potent ligands having sufficient affinity to a given receptor, a saturation will take place after a while, i.e., all receptors will be bound by an appropriate ligand. Because under some conditions, ligands that bind a receptor will enter the structured illumination field, fluorescence signals will be created over time, which however will decrease in intensity after a while due to photobleaching.

Preferably, in said method a physico-chemical response is evoked by the binding or dissociation reaction between the two or more molecules, which biochemical response is, optionally, recorded simultaneously.

One example for such embodiment would be a biochemical reaction evoked by binding of a ligand agonist labeled with a fluorophore to a given receptor (a reaction that can be recorded with the methods according to the present invention). Such receptor could be, for example, a ligand-dependent calcium channel, which changes its conformation after binding of the ligand. The physico-chemical response thus evoked is, for example, a receptor-activated calcium influx into the intracellular medium. This influx can be recorded by means of calcium sensitive dyes, i.e., fluorescent dyes that can respond to the binding of Ca²⁺ions by changing their fluorescence properties. The respective signals can be recorded simultaneously with the binding reaction. Another example is an endocytosis reaction evoked by ligand-activated receptors. Clathrin coated pit formation, its endocytosis and the trafficking of the resulting vesicle can then be followed optically in real time following a receptor activation event induced by ligand binding.

Furthermore, a method of determining the binding behavior or the dissociation behavior of at least one molecule with respect to another molecule is provided, said method encompassing a method as described above.

Likewise preferably, a method of screening a library of molecules in order to find a candidate molecule that binds to, or dissociates from, at least one predetermined other molecule is provided, said method encompassing a method as described above.

In this embodiment, a library of molecules labeled, e.g., with a fluorophore, is screened against a given receptor molecule which can be recombinantly fused to a different fluorophore or quencher. Such library can comprise anything between 3 molecules and 10¹² molecules or higher.

Likewise preferably, a method of comparing the binding behavior or the dissociation behavior of two or more candidate molecule against at least one predetermined molecule is provided, said method encompassing a method of any of the aforementioned claims, and in which method the two or more candidate molecules are labeled with different optical probes, or one is labeled and one is not labeled.

In this embodiment, a competitive binding assay can be established, in which for example the binding behavior of a labeled first ligand against a given receptor is compared to the binding behavior of a second ligand, which is, e.g., non-labeled, or labeled with another label. Likewise, in one embodiment, the first ligand is, for example, an agonist to said receptor, whereas the second ligand is an antagonist, e.g., a receptor-specific antibody.

### REFERENCES THE CONTENT OF WHICH IS INCORPORATED HEREIN

Winckler P et al, Identification and super-resolution imaging of ligand-activated receptor dimers in live cells Sci. Rep., 3 (2013) 2387

### OTHER REFERENCES

Sharonov et al., Wide-field subdiffraction imaging by accumulated binding of diffusing probes, PNAS 103 50 (2006) 18911

Giannone G et al., Dynamic Superresolution Imaging of Endogenous Proteins on Living Cells at Ultra-High Density, Biophys J. 2010 Aug. 9; 99(4): 1303-1310.

Bobroff N, Position measurements with a resolution and noise-limited instrument. Rev. Sci. Instrum. 1986; 57:1152-1157.

Thompson R E et al., Precise nanometer localization analysis for individual fluorescent probes. Biophys J. 2002 May; 82(5):2775-83.

### BRIEF DESCRIPTION OF THE EXAMPLES AND FIGURES

Additional details, features, characteristics and advantages of the object of the invention are disclosed in the subclaims, and the following description of the respective figures and examples, which, in an exemplary fashion, show preferred embodiments of the present invention. However, these drawings should by no means be understood as to limit the scope of the invention.

### EXPERIMENTS

Live Cell Super-Resolution Imaging of Functional Membrane EGFRs

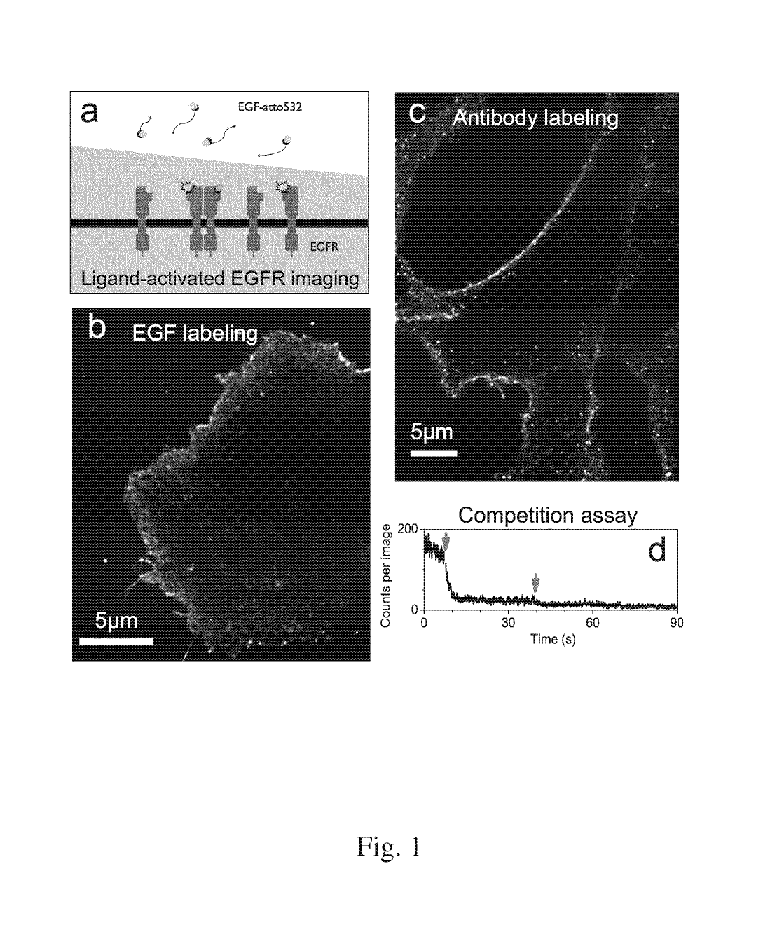

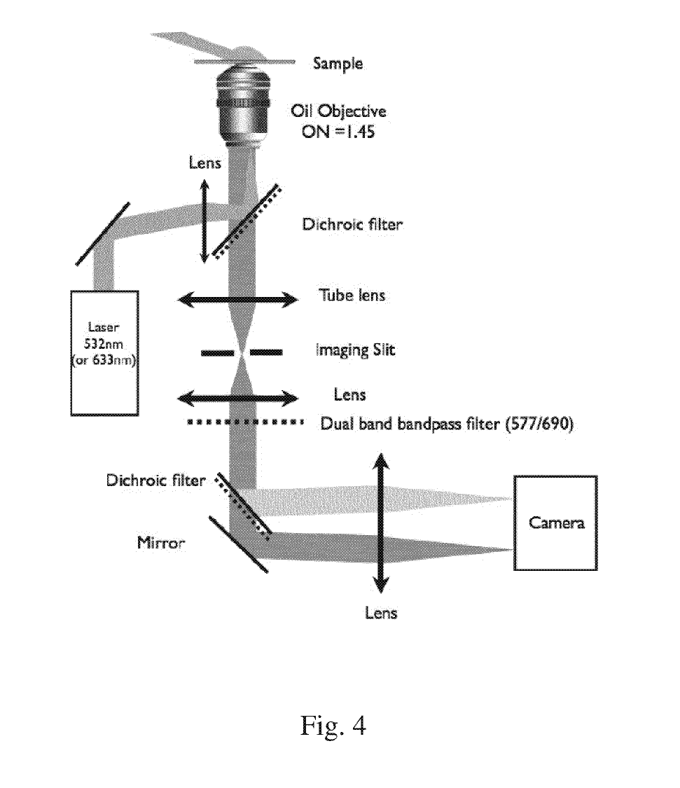

The observation of ligand-activated membrane EGFR dimers by single molecule FRET requires first that specific imaging of functional EGFRs newly activated by EGF can be obtained with high resolution at the cell membrane. To this aim, we designed a two-color super-resolution microscope based on the principle of PAINT/uPAINT. uPAINT relies on stochastically labeling in real-time of target biomolecules by fluorescent probes, and simultaneously recording their localization and dynamics on the cell membrane at the single molecule level using oblique illumination (FIG. 1a). The two-colors optical setup is built around a home-made dual-view system operating with single charge-coupled device camera (Methods and FIG. 4).

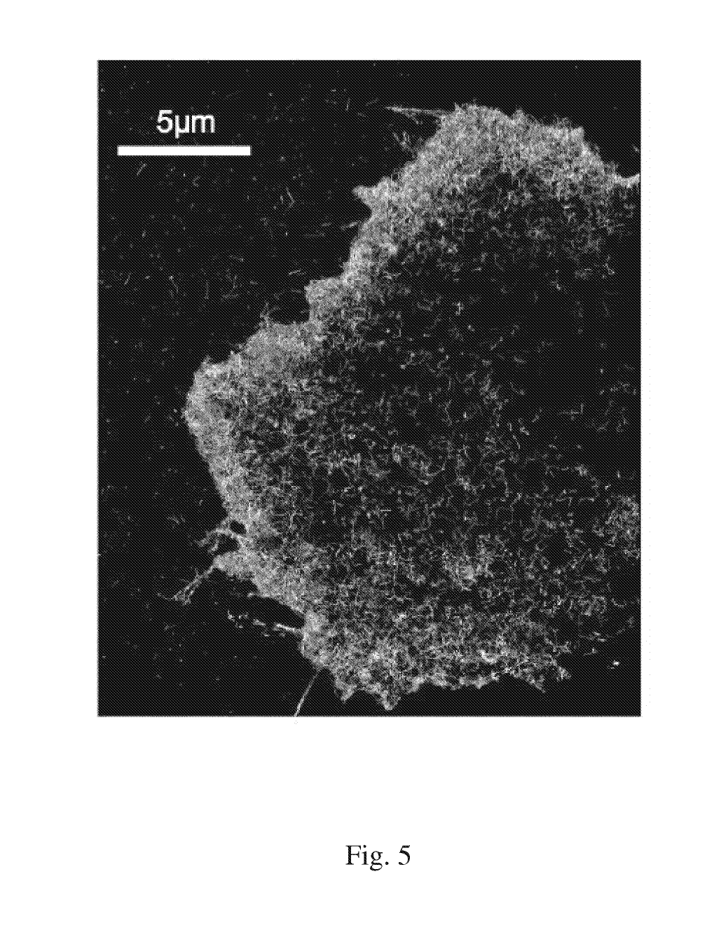

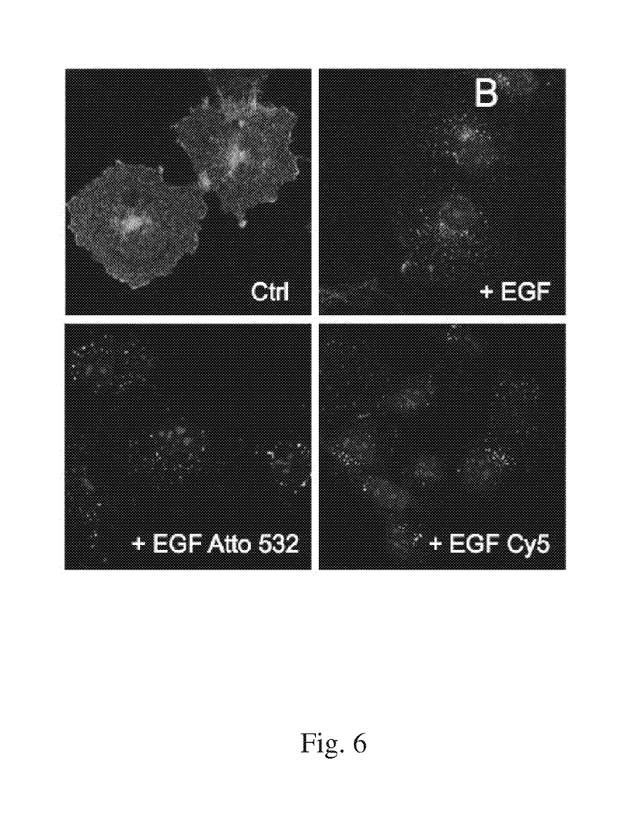

In a first experiment, live COS7 cells starved from growth factors overnight are illuminated with a 532 nm laser beam. Immediately after the beginning of recording, fluorescent EGF-Atto532 is introduced at low concentration (0.4 nM) in the imaging solution. Fluorescence images (8000 consecutive CCD frames) are recorded in the green detection channel with an integration time of 50 ms. The excitation beam angle is set to produce an inclined sheet of light above the glass slide with an illumination thickness of ˜2 μm at the center of the field of view. In those conditions, ligands newly bound to their target membrane receptor are efficiently illuminated while unbound fluorescent molecules freely diffusing in solution are mainly not illuminated. In addition, unbound molecules diffusing close to the cell membrane spend statistically at most two consecutive frames in the oblique illumination beam. Such unwanted events are rejected from the analysis performed. Continuous labeling and bound ligand photobleaching ensures sparse single molecule detection at the surface of the cells in each camera frame. Single molecule localizations are obtained with sub-diffraction precisions following image analysis (Methods). FIG. 1b shows a reconstructed image of endogenous EGFRs activated by EGF-Atto532 binding. The image is reconstructed from 1.6 10⁵ EGFR localizations belonging to ˜10⁴ single molecule trajectories. In this example, the selective imaging of activated EGF receptors with super-resolution is demonstrated (FIG. 5). Functionality of fluorescent ligand was controlled by observing that EGFR internalization occurs within minutes following binding of fluorescent EGF (FIG. 6).

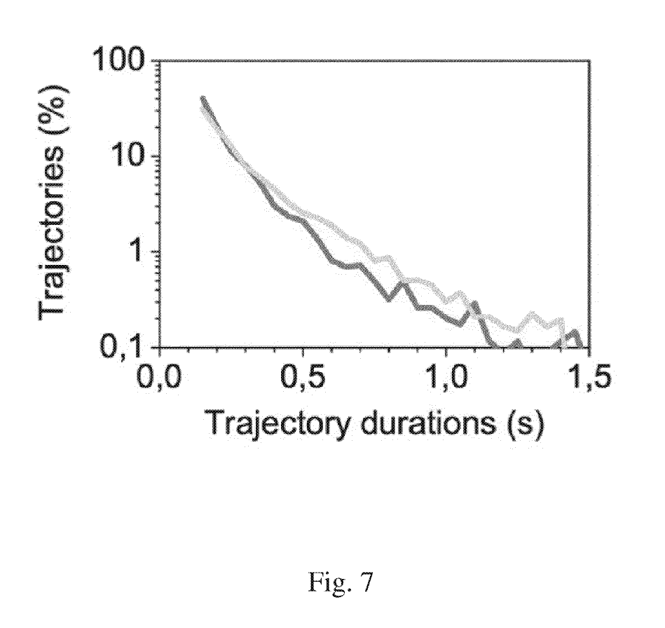

Noteworthy, the detection of each individual EGFR starts at the time an EGF binds the receptor. Thus an EGFR has to be at the membrane to be accessed, excluding any receptor complex localized just beneath the membrane in an early endosome. Receptor activation is captured with one imaging frame resolution (50 ms) and the detection of the activated receptors lasts until the fluorophore photobleaches. Such real-time imaging of agonist activation of single receptors allows the selective imaging of activated receptors with super-resolution. (typically in one second, see FIG. 7).

In our imaging conditions, this bleaching time being shorter than the lifetime of the activated receptors at the membrane before endocytosis, the super-resolution images exclusively display EGFRs that are present at the membrane. Capturing receptors in their early states following ligand binding would not be possible with photo-activation based super-resolution methods since fluorophore photoactivation and ligand binding processes are not time-correlated.

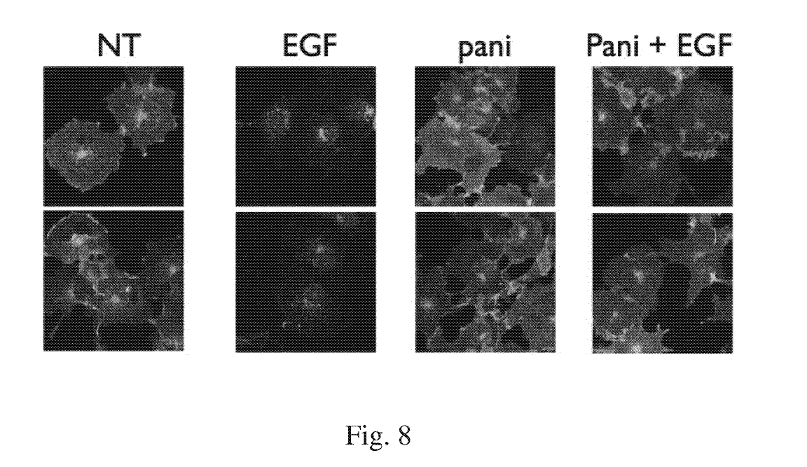

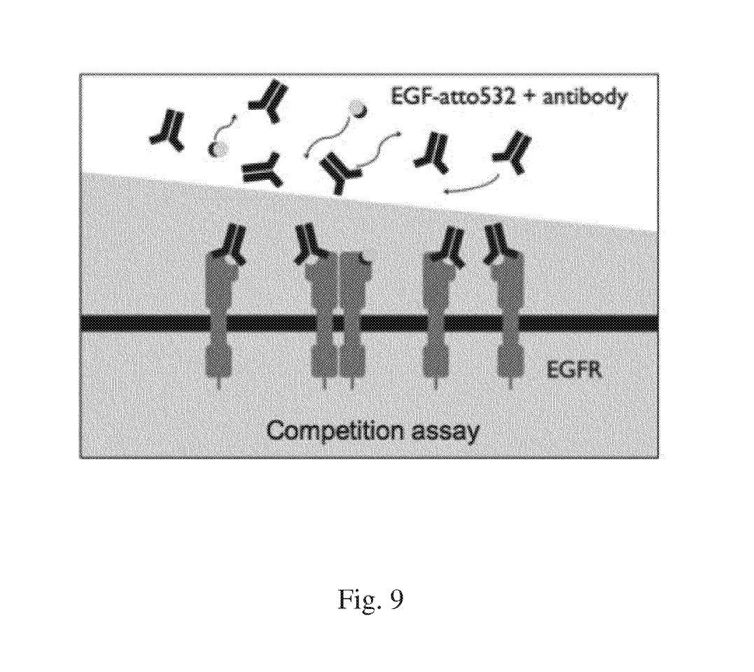

We next designed a live cell competition assay to evaluate the specificity of EGF-Atto532 labeling. We used panitumumab, a human monoclonal antibody highly specific to EGFRs which impedes EGF binding (FIG. 8). When a red fluorescent dye, Atto-647N excited with a 633 nm He-Ne laser was coupled to panitumumab (see methods), fluorescent panitumumab binding to EGFRs are detected in the red imaging channel producing uPAINT super-resolved images (FIG. 1c) akin to EGF labeling (FIG. 1b). In addition, if a uPAINT acquisition is started with EGF-Atto532 as in FIG. 1b and panitumumab is added in excess after a few seconds of recording (FIG. 9), then a dramatic drop of the number of fluorescent EGFR detected is observed (FIG. 1d). All together, these experiments indicate that fluorescent EGF labeling is highly specific and allow imaging functional and newly activated EGFRs with high resolution.

Live Cell Super-resolution Imaging of Dimers of Ligand-activated EGFRs

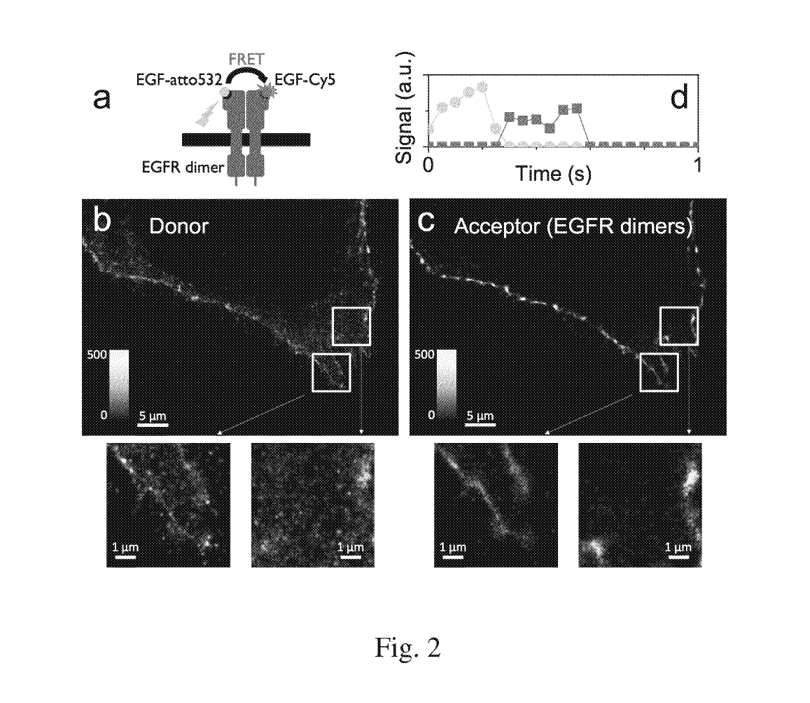

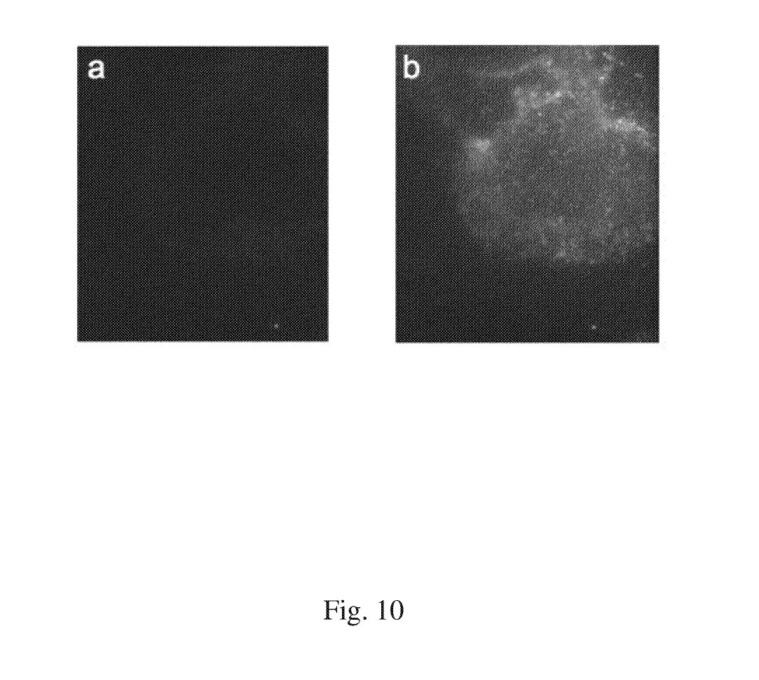

The super-resolved image presented in FIG. 1b displays the entire EGFR population localizations found at the membrane of live cells immediately after EGF activation, without distinction about the monomeric or multimeric state of the receptors. In particular, although present, EGFR dimers cannot be distinguished from isolated receptor in such images. In order to obtain images of EGFR dimers with super-resolution, we present in the following FRET experiments performed with EGF-Atto532 and EGF-Cy5 introduced in the imaging medium at equal concentration (˜0.2 nM). We chose Atto532 and Cy5 as a FRET pair, for their relatively large Forster radius, estimated to ˜65 Å. In order to identify FRET events (FIG. 2a), a single excitation laser (532 nm) was used and the images of the green and red camera channels were simultaneously recorded. In these illumination conditions, EGF-Atto532 is excited efficiently and detected solely in the green channel while EGF-Cy5 does not produce detectable signals when used alone (FIG. 10).

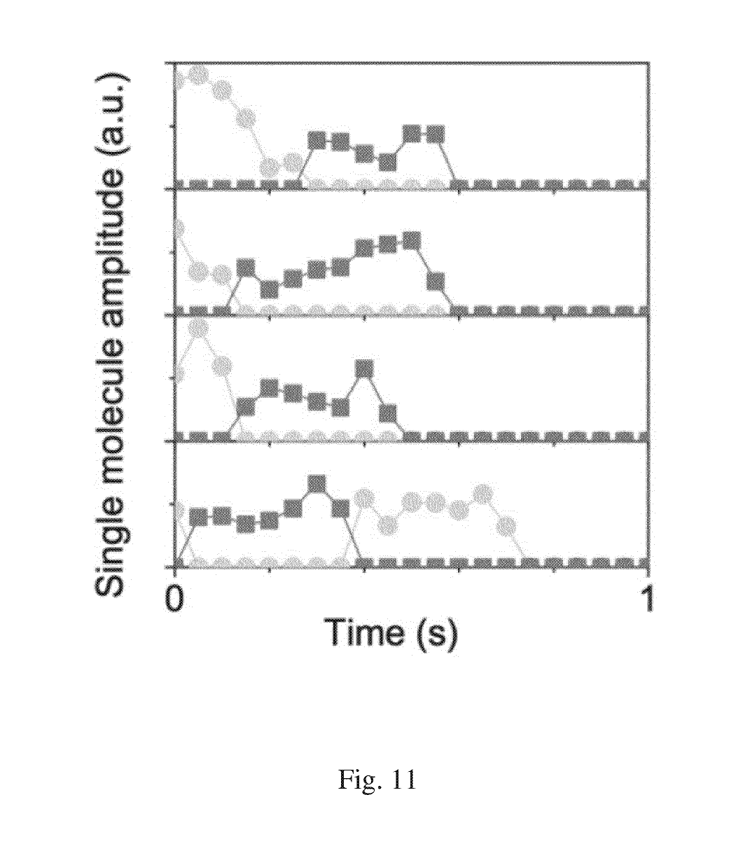

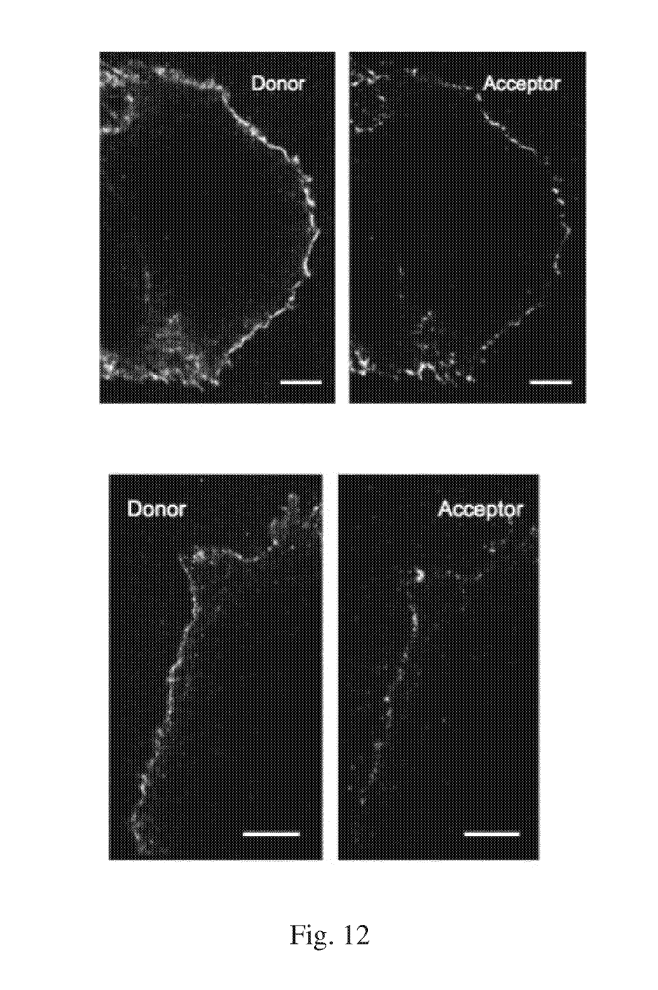

Gathering single molecule localizations recorded in the donor channel a super-resolved image of EGF activated EGFRs is obtained (FIG. 2b) (from 42,541 localizations corresponding to 7,078 single molecule trajectories), giving similar information than in FIG. 2b where EGFR monomers and multimers could be distinguished. Importantly, fluorescent spots are also recurrently observed on the cell surface in the acceptor channel. They originate from single molecule FRET occurring between EGFR dimers activated by an EGF-Atto532 and an EGF-Cy5 (FIG. 2a). This is further evidenced by the observation of anti-correlated signal detections in corresponding positions of the donor and acceptor channels (FIG. 2c and FIG. 11). Collecting the single molecule localizations obtained by image analysis in the acceptor channel, super-resolved images of EGF activated dimer EGFRs are reconstructed as shown on FIG. 2d (from 18,481 localizations corresponding to 3,350 trajectories) and on FIG. 12. Interestingly, the content of the acceptor channel is exclusively constituted by the subpopulation of EGFR dimers activated by two EGF molecules (an EGF-Atto532 and an EGF-Cy5). In the donor channel, this subpopulation was also present (in the form of dimers labeled by two EGF-Atto532) but was undistinguishable from other activated EGFRs populations (including EGFR monomers). It is thus possible to compare the images reconstructed from the two channels in order to extract the specific localization of newly activated EGFR dimers from the entire population. Hence, we demonstrate, on the EGF/EGFR, system that FRET can be used in super-resolution to selectively image receptor dimers.

Membrane Diffusion Properties of Ligand Activated EGFRs Dimers

We next used single molecule tracking to study the diffusion properties of EGFR at the cell membrane. Dimers mobility could thus be compared to that of the mixed population found in the donor channel with high statistics, to thus demonstrated that the concept that FRET can be used in super-resolution imaging to selectively study the spatio-temporal dynamics of a receptor subpopulation consisting of receptor dimers.

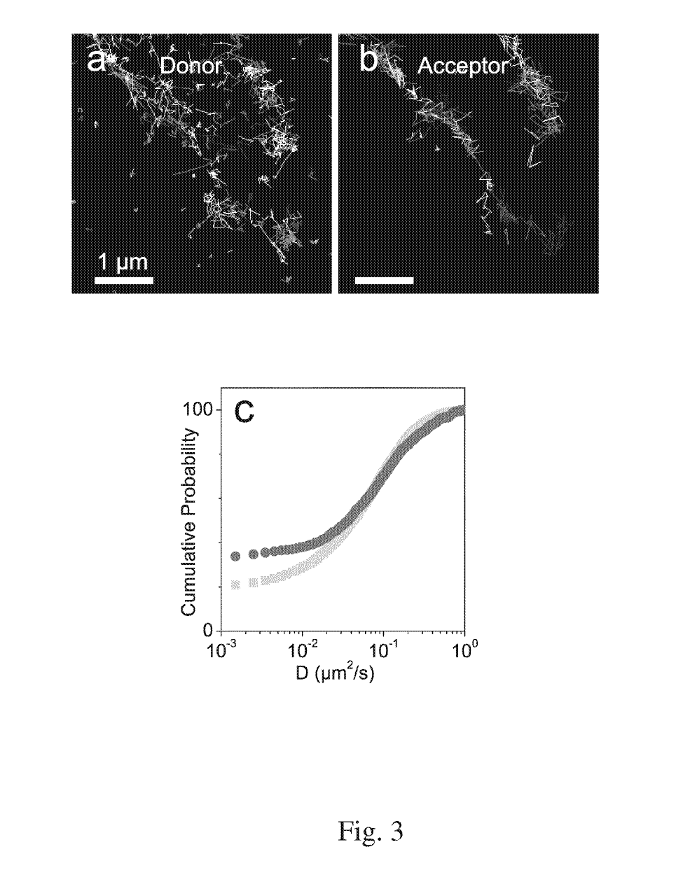

We analyzed the trajectories lasting more than 200 ms detected in the acceptor (n=2025) and donor (n=4530) channels. Examples of such trajectories are displayed in FIGS. 3a and 3b. We computed the mean square displacement and measured the slopes at the origin to extract the instantaneous diffusion coefficient, D, of each tracked entity (see Methods). Molecules with diffusion constant <7×10⁻³μm²/s were considered as immobile within our resolution. FIG. 3c presents the cumulative distribution of D values obtained on a single cell for EGFR dimers and for the entire population of activated EGFRs imaged in the donor channel. Both distributions present an heterogeneity of diffusion coefficients (ranging from highly mobile to immobile molecules) suggesting that the heterogeneity observed in the donor channel (green data point in FIG. 3c) is not primarily due to the multiplicity of EGFR multimeric compositions. Finally, the proportion of immobile dimers for the pure dimer population found by FRET (red data points in FIG. 3c) is more pronounced than for the entire population of activated EGFRs imaged in the donor channel. Activated EGFR monomers are thus likely to be more mobile than activated dimers. The frequent immobilizations undergone by the dimer population might be in part the consequence of dimer trapping in preformed endocytotic coated pits.

Methods

Labeling of the EGF and Antibody with Fluorescent Dyes

Recombinant mouse EGF (R&D systems) was conjugated with Atto532-NHS-ester (Atto-Tec) or Cy5-NHS-ester (Amersham Bioscience) by using modified versions of the manufacturers' procedures. Briefly, 100 μg of EGF (1mg/mL) were incubated with 10 μL of Atto532-NHS-ester (or Cy5-NHS-ester) (5 mM) in the presence of NaHCO3 (0.1 M) for 2 h at room temperature. Separation of labeled ligands from unbounded dyes was performed in size-exclusion columns (Sephadex G25; Pharmacia, New Market, N.J.). As mouse-EGFs have only one reactive amino residue, the protein/dye ratio was 1:1. Panitumumab antibodies were labeled with Atto647N-NHS-ester (Atto-Tec) using the same protocol (antibodies: dye ratio of about 1:1).

Sample Preparation

COS 7 cells are cultured in DMEM(Gibco) with 10% FBS, The day before the experiment, cells are detached with trypsin/EDTA and platted on clean coverslips. After few hours, cells are washed and cultured in serum free condition. Experiments are performed in Ringer (in mM: 150 NaCl, 5 KCl₂ CaCl₂ MgCl₂, 10 HEPES, 11 Glucose, pH 7.4) with 1 mg/mL bovine serum albumin to reduce non-specific ligand adsorption. Before acquisition, the sample is incubated with a solution containing a low concentration of fluorescent beads to provide upon unspecific adsorption on the coverslip immobile reference objects used to correct long-term mechanical instabilities of the microscope. Then, the coverslip is mounted on an open chamber and 300 μl of Ringer solution is added onto the cells. At the beginning of the camera recording, 10 μL of fluorescent ligands are added (final concentration is about 0.4 nM).

Two-color uPAINT Setup

uPAINT acquisitions are performed on a custom-made dual-color microscope (FIG. 4), It is based on an inverted microscope (Olympus) equipped with a 100×1.45 NA objective. Fluorophores are excited in wide-field oblique illumination by tilting a collimated laser beam in the object focal plane of the imaging lens which focuses the beams in the back focal plane of the objective. The resulting angle at the sample is set to ˜5°. This allowed to image individual fluorescent ligands which have bound to the cell surface while not illuminating the molecules in the above solution. The angle was chosen to obtain an illumination thickness of ˜2μm in the center of the field.

Atto532 dyes were excited by a 532 nm laser solid state laser (Compass 415 M, Coherent). Cy5 dyes or Atto647N dyes were excited by a 633 nm HeNe (Thorlabs). A dichroic filter (Semrock FF655-Di01) placed in the infinity detection path combined with a double bandpass emission filter (Semrock FF01-577/690) allows a spectral selection of the fluorescence signals in the donor (Atto532) and acceptor (Cy5) channels obtained simultaneously in two separated images on the EM-CCD camera (QuantEM512SC, Photometrics). A slit (˜5 mm width) is placed in the imaging plane of the tube lens to avoid overlap of the donor and acceptor images on the CCD chip. Images are acquired at 20 frames/s rates. Excitation intensities were ˜2kW/cm² and ligand concentrations were adjusted in order to have a constant pool of ˜0.5 mol/μm² fluorescent molecules. We used fluorescent beads adsorbed on the glass coverslips as immobile fiduciary markers to correct for long-term mechanical instabilities of the microscope.

Control uPAINT experiments with EGF-Cy5 alone showed no single molecule detections in either detection channel using 532 nm laser excitation. In addition, when EGF-Atto532 is introduced alone, no single molecule detection can be detected in the red channel.

Single Molecule Segmentation and Tracking

A typical single cell, acquired with the uPAINT microscope setup and protocol described above, leads to a set of 8,000 images that further need to be analysed in order to extract molecule localization and dynamics. The center coordinates of single molecule fluorescent spots were localized in each image frame with sub-wavelength accuracy and tracked over time. Under the experimental conditions described above, the localization accuracy of the whole system was quantified to ˜40 nm (full width at half maximum). Super-resolved images were computed by cumulating single molecule coordinates for all frames, using the same intensity for each localization.

To analyze the trajectories we used the mean squared displacement MSD computed as:

$$MSD ( t = n \cdot Δ t ) = \frac{{\sum }_{i = 1}^{N - n} {( {x}_{i + n} - {x}_{i} )}^{2} + {( {y}_{i + n} - {y}_{i} )}^{2}}{N - n}$$

where xᵢ and yᵢ are the coordinates of the label position at time i·Δt. We defined the measured diffusion coefficient D as the slope of the affine regression line fitted to the n=1 to 4 values of the MSD(n·Δt). Short-trajectories (<4 points), were filtered out. Immobile trajectories were defined as trajectories with D<0.007 μm²s⁻¹, corresponding to molecules which explored an area inferior to the one defined by the image spatial resolution (˜0.05μm)² during the time used to fit the initial slope of the MSD.

### FIGURES

FIG. 1. Live cell super-resolution imaging of functional membrane EGFRs newly activated by their ligand

(a) Principle of the super-resolution method. Oblique illumination (light green) does not excite EGF ligands in solution. (b) uPAINT image of EGFR labeled by EGF-Atto532 acquired on live cells. (c) Same experiment performed using Panitumumab-Atto647N. (d) Competition assay showing specificity of EGF-Atto532 labeling: number of fluorescent EGF detected per frame (50 ms) on the cell membrane during a uPAINT acquisition using EGF-Atto532. After ˜8s and 38s (red arrows), unlabeled Panitumumab was added in 100-fold excess (40 nM) compared to EGF.

FIG. 2. Live cell super-resolution imaging of membrane EGFR dimers based on single-molecule FRET

Dual color uPAINT imaging of EGFR was performed using a 1:1 mix of EGF-Atto532 and EGF-Cy5 under 532 nm laser excitation. (a) Schematics of single molecule FRET between two fluorescent ligands bound on a EGFR dimer. (b) Donor channel: super-resolved image of EGFR labeled by EGF-Atto532 as in FIG. 1b. (c) Acceptor channel: super-resolved image of EGF activated dimer EGFRs obtained by single molecule FRET. (d) Signature of single molecule FRET: anti-correlated fluorescence signals detected by single molecule fitting in the donor (green line) and acceptor (red line) channels, in corresponding positions. Insets in (b) and (c) represents zooms of highlighted regions showing preferential cell edge localization of the dimers.

FIG. 3. Membrane dynamics of EGFR dimers based on single molecule tracking of the FRET acceptor signals
(a) and (b) Color coded trajectories lasting more than 200 ms found in one of the highlighted regions of FIG. 2b and c respectively. (c) Cumulative distribution of D values obtained on a single cell for EGFR dimers alone (red) and for the entire population of EGFR imaged in the donor channel (green).

FIG. 4. Schematics of the 2-color uPAINT optical setup

FIG. 5. Trajectories of ligand activated EGFRs

Color-coded trajectories lasting more than 4 points (200 ms) corresponding to the ligand activated EGFRs displayed in FIG. 1a.

FIG. 6. Functionality of the fluorescent EGF

Immunocytochemistry. Live COS7 cells were seeded in a 96 well imaging plate and starved for 24 hours before addition of vehicle or 100 ng/ml of bare EGF (Roche), Atto532-EGF or Cy5-EGF for 15 minutes. Cells were then fixed with 3.7% of formaldehyde for 10 minutes and permeabilized in 0.5% Triton X-100 for 5 minutes. Staining was performed using a specific primary antibody (anti-EGFR, Zymed) overnight at 4° C. and a fluorescent secondary antibody conjugate (Alexa Fluor 488 anti-mouse IgG, Molecular Probes, Life Technologies) 1 hour at room temperature in order to reveal the localization of EGFR upon addition of EGF. Nuclei were stained with Hoechst 33258 (Molecular Probes, Life technologies). Image acquisition was done using the LSM 510 Meta microscope (Zeiss, France) Intracellular localization of EGFR induced by EGF, Atto532-EGF or Cy5-EGF was indistinguishable, validating the functionality of Atto532-EGF or Cy5-EGF.

FIG. 7. Distribution of the single molecule trajectory durations detected on live COS 7 cells in FRET experiments

Green: EGFRs labeled by Atto532-EGF detected in the donor channel, Red: EGFR dimers detected by single molecule FRET (Cy5-EGF emission) detected in the acceptor channel. Only trajectories lasting more than 150 ms are taken into account. N=6650 (donor channel), and N=3447 (acceptor channel).

FIG. 8. Panitumumab prevents EGF binding to EGFRs

Live COS7 cells were seeded in a 96 well imaging plate and starved for 24 hours before addition of vehicle or 200 μg/ml of panitumumab (Amgen) for 6 hours. EGF (100 ng/ml) was added 15 minutes before fixation with paraformaldehyde 3.7% and permeabilization with 0.5% Triton X-100. Anti-EGFR immunostaining was performed as explained in FIG. 6. Image acquisition was done using the LSM 510 Meta microscope (Zeiss, France). Images indicate that EGF-induced EGFR internalization is blocked by the addition of panitumumab.

FIG. 9. Schematics illustrating the principle of the competition experiment presented in FIG. 1d.

After ˜8s following the beginning of a uPAINT experiment using EGF-Atto532, unlabeled Panitumumab is added in 100-fold excess (40 nM) compared to EGF-Atto532.

FIG. 10. Single molecule FRET control experiment

When COS7 cells are labeled with Cy5-EGF alone, excitation at 532 nm does not produce detectable single molecule signals in the donor channel (a) while subsequent excitation at 633 nm reveals that EGFR bound Cy5-EGF have accumulated on the cell surface. A fluorescent bead adsorbed on the glass coverslips acting as fiduciary marker to correct for long-term mechanical instabilities of the microscope is visible on the two images.

FIG. 11. Single molecule FRET anti-correlated signals

Examples of single molecule anti-correlated signals detected by the fitting algorithm similar to that displayed on FIG. 2d: signals from the donor (green line) and acceptor (red line) channels, in corresponding positions.

FIG. 12, Live cell super-resolution imaging of membrane EGFR dimers based on singlemolecule FRET

Representative examples as shown on FIG. 2

1-26. (canceled)

27. A super-resolution imaging method comprising:
carrying out a (i) binding reaction, (ii) dissociation reaction, (iii) competitive binding reaction, or (iv) association reaction between two or more molecules in a biological sample; and
imaging the (i) binding reaction, (ii) dissociation reaction, (iii) competitive binding reaction, or (iv) association reaction between two or more molecules in a biological sample by super-resolution imaging,
wherein the reaction as such causes an event that forms the basis for identification and localization-based super-resolution microscopy of the reaction.
